(19) United States
(12) Patent Application Publication
(10) Pub. No.: US 2013/0096035 A1
(43) Pub. Date: Apr. 18, 2013

(54) SELF-SUSTAINED FLUIDIC DROPLET CASSETTE AND SYSTEM FOR BIOCHEMICAL ASSAYS

(71) Applicants: Tza-Huei Wang, Timonium, MD (US); Seungkyung Park, Pikesville, MD (US); Samuel Yang, Ellicott City, MD (US); Yi Zhang, Baltimore, MD (US)

(72) Inventors: Tza-Huei Wang, Timonium, MD (US); Seungkyung Park, Pikesville, MD (US); Samuel Yang, Ellicott City, MD (US); Yi Zhang, Baltimore, MD (US)

(73) Assignee: The Johns Hopkins University, Baltimore, MD (US)

(21) Appl. No.: 13/805,625

(22) PCT Filed: Jul. 26, 2011
(86) PCT No.: PCT/US11/45363

§ 371 Date: Dec. 19, 2012

Related U.S. Application Data
(60) Provisional application No. 61/367,690, filed on Jul. 26, 2010.

(51) Int. Cl. C12Q 1/68

(52) U.S. Cl. CPC C12Q 1/686
USPC 506/39; 435/287.2

(57) ABSTRACT

A fluidic cartridge for biochemical assays includes a cartridge body defining a first droplet region and a second droplet region with a droplet restraining barrier therebetween. The droplet restraining barrier has a gap between the first and the second droplet regions. The fluidic cartridge also includes a first droplet dispensed in the first droplet region. The first droplet includes a plurality of magnetic particles dispersed therein. The fluidic cartridge also includes a second droplet disposed in the second droplet region. The plurality of magnetic particles are sufficiently small to be drawn through the gap between the first and second droplet regions when compelled by an applied magnetic field, and the first droplet is restrained by the restraining barrier while the plurality of magnetic particles are drawn through the gap. A biochemical assay system includes a stage adapted to receive a fluidic cartridge, and a magnetic control assembly that includes a magnet. The magnet of the magnetic control assembly is movable to direct motion of magnetic particles contained within the fluidic cartridge.

### CROSS-REFERENCE OF RELATED APPLICATION

This application claims priority to U.S. Provisional Application No. 61/367,690 filed Jul. 26, 2010, the entire contents of which are hereby incorporated by reference.

This invention was made with Government support of Grant No. U54 AI057168, awarded by the Department of Health and Human Services, The National Institutes of Health (NIH) and Grant No. ECCS-0725528, awarded by the National Science Foundation (NSF). The U.S. Government has certain rights in this invention.

### BACKGROUND

1. Field of Invention

The field of the currently claimed embodiments of this invention relates to fluidic cassettes and systems for biochemical assays.

2. Discussion of Related Art

During the past few decades, with the advancement of micro-electromechanical systems (MEMS) and microfluidic technologies, great effort has been put into the development of miniaturized platforms for various biological and chemical analyses. For example, see the following:
Vilkner, T., Janasek, D. & Manz, A. Micro total analysis systems. Recent developments. Analytical Chemistry 76, 3373-3385 (2004);
Abgrall, P. & Gue, A. M. Lab-on-chip technologies: making a microfluidic network and coupling it into a complete microsystem—a review. Journal of Micromechanics and Microengineering 17, R15-R49 (2007);
Craighead, H. Future lab-on-a-chip technologies for interrogating individual molecules. Nature 442, 387-393 (2006);
Daw, R. & Finkelstein, J. Lab on a chip. Nature 442, 367-367 (2006);
deMello, A. J. Control and detection of chemical reactions in microfluidic systems. Nature 442, 394-402 (2006);
El-Ali, J., Sorger, P. K. & Jensen, K. F. Cells on chips. Nature 442, 403-411 (2006). Janasek, D., Franzke, J. & Manz, A. Scaling and the design of miniaturized chemical-analysis systems. Nature 442, 374-380 (2006);
Psaltis, D., Quake, S. R. & Yang, C. H. Developing optofluidic technology through the fusion of microfluidics and optics. Nature 442, 381-386 (2006); and
Yager, P., Edwards, T., Fu, E., Helton, K., Nelson, K., Tam, M. R. & Weigl, B. H. Microfluidic diagnostic technologies for global public health. Nature 442, 412-418 (2006).

Advantages of these micro-machined or micro-fabricated devices can include fast analysis, low sample and reagent consumption, low cost, high portability and disposability. They are also well recognized to have great potential for automation and integration into micro total analysis systems (μTAS) that include sample preparation, reaction and detection modules that are combined together through microfluidic transportation and/or separation units (Dittrich, P. S., Tachikawa, K. & Manz, A. Micro total analysis systems. Latest advancements and trends. Analytical Chemistry 78, 3887-3907 (2006); Auroux, P. A., Iossifidis, D., Reyes, D. R. & Manz, A. Micro total analysis systems. 2. Analytical standard operations and applications. Analytical Chemistry 74, 2637-2652 (2002); Reyes, D. R., Iossifidis, D., Auroux, P. A. & Manz, A. Micro total analysis systems. 1. Introduction, theory, and technology. Analytical Chemistry 74, 2623-2636 (2002)). Although conventional continuous flow microfluidic systems have made significant progress towards integrating multiple tasks onto a single miniaturized platform for biochemical detection, most of such platforms are not available for point-of-care assays, mainly because they involve many complicated micro-fluidic functional units, such as micro-valves, micro-pumps and interface connections. Moreover, conventional microfluidic platforms often require bulky peripheral accessories and controllers that are not practically portable. Droplet-based microfluidic devices have recently attracted more and more attention. For example, see the following:
Rane, T. D., Puleo, C. M., Liu, K. J., Zhang, Y., Lee, A. P. & Wang, T. H. Counting single molecules in sub-nanolitre droplets. Lab on a Chip 10 (2010);
Fan, S. K., Hsieh, T. H. & Lin, D. Y. General digital microfluidic platform manipulating dielectric and conductive droplets by dielectrophoresis and electrowetting. Lab on a Chip 9, 1236-1242 (2009);
Teh, S. Y., Lin, R., Hung, L. H. & Lee, A. P. Droplet microfluidics Lab on a Chip 9, 3604-3604 (2009);
Guttenberg, Z., Muller, H., Habermuller, H., Geisbauer, A., Pipper, J., Felbel, J., Kielpinski, M., Scriba, J. & Wixforth, A. Planar chip device for PCR and hybridization with surface acoustic wave pump. Lab on a Chip 5, 308-317 (2005);
Hsieh, T.-M., Zhang, Y., Pipper, J. & Neuzil, P. PCR by moving a free droplet over different temperature zones. microTAS 2006 conference proceeding (2006);
Pipper, J., Inoue, M., Ng, L. F. P., Neuzil, P., Zhang, Y. & Novak, L. Catching bird flu in a droplet. Nat. Med. 13, 1259-1263 (2007);
Pipper, J., Zhang, Y., Neuzil, P. & Hsieh, T. M. Clockwork PCR including sample preparation. Angew. Chem. 47, 3900-3904 (2008);
Lehmann, U., Vandevyver, C., Parashar, V. K. & Gijs, M. A. M. Droplet-based DNA purification in a magnetic lab-on-a-chip. Angew. Chem. 45, 3062-3067 (2006);
Diehl, F., Li, M., He, Y. P., Kinzler, K. W., Vogelstein, B. & Dressman, D. BEAMing: single-molecule PCR on microparticles in water-in-oil emulsions. Nat. Methods. 3, 551-559 (2006);
Margulies, M., Egholm, M., Altman, W. E., Attiya, S., Bader, J. S., Bemben, L. A., Berka, J., Braverman, M. S., Chen, Y. J., Chen, Z. T., Dewell, S. B., Du, L., Fierro, J. M., Gomes, X. V., Godwin, B. C., He, W., Helgesen, S., Ho, C. H., Irzyk, G. P., Jando, S. C., Alenquer, M. L. I., Jarvie, T. P., Jirage, K. B., Kim, J. B., Knight, J. R., Lanza, J. R., Leamon, J. H., Lefkowitz, S. M., Lei, M., Li, J., Lohman, K. L., Lu, H., Makhijani, V. B., McDade, K. E., McKenna, M. P., Myers, E. W., Nickerson, E., Nobile, J. R., Plant, R., Puc, B. P., Ronan, M. T., Roth, G. T., Sarkis, G. J., Simons, J. F., Simpson, J. W., Srinivasan, M., Tartaro, K. R., Tomasz, A., Vogt, K. A., Volkmer, G. A., Wang, S. H., Wang, Y., Weiner, M. P., Yu, P. G., Begley, R. F. & Rothberg, J. M. Genome sequencing in microfabricated high-density picolitre reactors. Nature 437, 376-380 (2005);
Zhang, J. H., Cheng, Z. J., Zheng, Y. M. & Jiang, L. Ratchet-induced anisotropic behavior of superparamagnetic microdroplet. Applied Physics Letters 94 (2009);
Shastry, A., Case, M. J. & Bohringer, K. F. Engineering surface roughness to manipulate droplets in microfluidic systems. MEMS 2005 Miami: Technical Digest, 694-697 (2005);
Velev, O. D., Prevo, B. G. & Bhatt, K. H. On-chip manipulation of free droplets. Nature 426, 515-516 (2003);
Ohashi, T., Kuyama, H., Hanafusa, N. & Togawa, Y. A simple device using magnetic transportation for droplet-based PCR. Biomedical Microdevices 9, 695-702 (2007); and
Zhang, Y., Bailey, V., Puleo, C. M., Easwaran, H., Griffiths, E., Herman, J. G., Baylin, S. B. & Wang, T. H. DNA methylation analysis on a droplet-in-oil PCR array. Lab on a Chip 9, 1059-1064 (2009).

One major form of droplet microfluidic devices manipulates droplets on an open surface. Such droplets are self-contained and function both as a reaction chamber and a fluid transportation unit (Guttenberg, Z., Muller, H., Habermuller, H., Geisbauer, A., Pipper, J., Felbel, J., Kielpinski, M., Scriba, J. & Wixforth, A. Planar chip device for PCR and hybridization with surface acoustic wave pump. Lab on a Chip 5, 308-317 (2005); Hsieh, T.-M., Zhang, Y., Pipper, J. & Neuzil, P. PCR by moving a free droplet over different temperature zones. microTAS 2006 conference proceeding (2006); Pipper, J., Inoue, M., Ng, L. F. P., Neuzil, P., Zhang, Y. & Novak, L. Catching bird flu in a droplet. Nat. Med. 13, 1259-1263 (2007); Pipper, J., Zhang, Y., Neuzil, P. & Hsieh, T. M. Clockwork PCR including sample preparation. Angew. Chem. 47, 3900-3904 (2008)). Many actuation methods have been developed to control the droplet movement, including passive actuation (Zhang, J. H., Cheng, Z. J., Zheng, Y. M. & Jiang, L. Ratchet-induced anisotropic behavior of superparamagnetic microdroplet. Applied Physics Letters 94 (2009); Shastry, A., Case, M. J. & Bohringer, K. F. Engineering surface roughness to manipulate droplets in microfluidic systems. MEMS 2005 Miami: Technical Digest, 694-697 (2005)) and active actuation, such as dielectrophoresis (Fan, S. K., Hsieh, T. H. & Lin, D. Y. General digital microfluidic platform manipulating dielectric and conductive droplets by dielectrophoresis and electrowetting. Lab on a Chip 9, 1236-1242 (2009); Velev, O. D., Prevo, B. G. & Bhatt, K. H. On-chip manipulation of free droplets. Nature 426, 515-516 (2003)), surface acoustic wave (Guttenberg, Z., Muller, H., Habermuller, H., Geisbauer, A., Pipper, J., Felbel, J., Kielpinski, M., Scriba, J. & Wixforth, A. Planar chip device for PCR and hybridization with surface acoustic wave pump. Lab on a Chip 5, 308-317 (2005)), electrowetting (Fan, S. K., Hsieh, T. H. & Lin, D. Y. General digital microfluidic platform manipulating dielectric and conductive droplets by dielectrophoresis and electrowetting. Lab on a Chip 9, 1236-1242 (2009)) or magnetic force (Hsieh, T.-M., Zhang, Y., Pipper, J. & Neuzil, P. PCR by moving a free droplet over different temperature zones. microTAS 2006 conference proceeding (2006); Pipper, J., Inoue, M., Ng, L. F. P., Neuzil, P., Zhang, Y. & Novak, L. Catching bird flu in a droplet. Nat. Med. 13, 1259-1263 (2007); Pipper, J., Zhang, Y., Neuzil, P. & Hsieh, T. M. Clockwork PCR including sample preparation. Angew. Chem. 47, 3900-3904 (2008); Lehmann, U., Vandevyver, C., Parashar, V. K. & Gijs, M. A. M. Droplet-based DNA purification in a magnetic lab-on-a-chip. Angew. Chem. 45, 3062-3067 (2006); Ohashi, T., Kuyama, H., Hanafusa, N. & Togawa, Y. A simple device using magnetic transportation for droplet-based PCR. Biomedical Microdevices 9, 695-702 (2007)). Among the actuation methods mentioned above, magnet-actuated droplets can have special advantages because of flexibility and ease of operation. The use of permanent magnets can replace external controlling units, hence greatly reducing fabrication and operation cost. Furthermore, this valve-less and pump-less microfluidic platform can be extremely useful for point-of-care sample preparation and analysis in a timely fashion due to their reduced complexity and high portability. In addition, the magnetic particles used for droplet actuation can also serve as carriers for biomolecules, such as using silica superparamagnetic particles (SSP) for nucleic acid binding and transfer. Despite its simplicity, there are still many factors, such as liquid to particle ratio (L/P ratio) and magnet moving speed, that affect the microfluidic control of droplets on the surface (Long, Z., Shetty, A. M., Solomon, M. J. & Larson, R. G. Fundamentals of magnet-actuated droplet manipulation on an open hydrophobic surface. Lab on a Chip 9 (2009)). In many applications, larger surface area, thus more magnetic particles are required for efficient biomolecule adsorption. However, such conditions may not favor microfluidic control of the droplet because the small L/P ratios prevent easily splitting the SSP from the droplet.

One major application of a nucleic-acid μTAS is to perform molecular diagnostics at the point of care, which is crucial for immediate clinical decisions and treatment. Infectious disease is the second most prevalent cause of death according to WHO (WHO Annex Table 2: Deaths by cause, sex and mortality stratum in WHO regions, estimates for 2002. The world health report 2004—changing history (2004)). Compared with conventional culture-based approaches, genetics-based molecular diagnostics have greatly reduced the turn-around time for the identification of infectious agents. However, current technologies are still limited to labor-intensive sample preparation and centralized laboratory operations, hindering the routine use of molecular diagnostic methods at patient sites or in low-resource environments. Another major cause of death is cancer (WHO Annex Table 2: Deaths by cause, sex and mortality stratum in WHO regions, estimates for 2002. The world health report 2004—changing history (2004)). The concept of personalized medicine, which is believed to be the key to cancer treatment, is becoming widely accepted (Nevins, J. R., Huang, E. S., Dressman, H., Pittman, J., Huang, A. T. & West, M. Towards integrated clinico-genomic models for personalized medicine: combining gene expression signatures and clinical factors in breast cancer outcomes prediction. Human Molecular Genetics 12, R153-R157 (2003); Ginsburg, G. S. & McCarthy, J. J. Personalized medicine: revolutionizing drug discovery and patient care. Trends in Biotechnology 19, 491-496 (2001)). Cancers may have different causes, rates of progression and responsiveness to pharmaco-radio-therapies or chemotherapies. As a result, each patient's disease might be very unique. Molecular biomarkers, such as DNA-based biomarkers, provide a patient's individualized information which can enable medical personnel to predict the rate and severity of cancers and tailor the treatment accordingly. More importantly, some of the molecular biomarkers indicate the risks of developing cancers. High-risk populations can therefore modify their lifestyle and take preventive therapies (Ginsburg, G. S. & McCarthy, J. J. Personalized medicine: revolutionizing drug discovery and patient care. Trends in Biotechnology 19, 491-496 (2001)). With personalized medicine becoming more and more popular, low-cost, easily accessible and fully functional molecular biomarker detection systems are much desired.

Although a great number of nucleic-acid-based assays are currently available, the polymerase chain reaction (PCR) remains the dominant technique due to its simplicity and high sensitivity. Many state-of-art bio-molecular assays still heavily rely on PCR or modified versions of PCR, such as real time PCR for gene quantification (Heid, C. A., Stevens, J., Livak, K. J. & Williams, P. M. Real time quantitative PCR. Genome Research 6, 986-994 (1996)), bridging PCR (Fedurco, M., Romieu, A., Williams, S., Lawrence, I. & Turcatti, G. BTA, a novel reagent for DNA attachment on glass and efficient generation of solid-phase amplified DNA colonies. Nucleic Acids Research 34 (2006)) and BEAMing PCR (Diehl, F., Li, M., He, Y. P., Kinzler, K. W., Vogelstein, B. & Dressman, D. BEAMing: single-molecule PCR on microparticles in water-in-oil emulsions. Nat. Methods. 3, 551-559 (2006)) for the ‘next generation’ of nucleic-acid sequencing (Shendure, J. & Ji, H. L. Next-generation DNA sequencing. Nature Biotechnology 26, 1135-1145 (2008)). Numerous studies have attempted to translate the ensemble PCR assay onto a microfluidic chip and have made great contributions to various aspects, including device fabrication and packaging techniques, materials, microfluidic architecture and handling, surface modification, etc. (Zhang, C. S., Xu, J. L., Ma, W. L. & Zheng, W. L. PCR microfluidic devices for DNA amplification. Biotechnology Advances 24, 243-284 (2006)). Other popular nucleic-acid amplification methods mainly fall into the category of isothermal amplification in which the reactions are performed by maintaining a constant temperature, hence bypassing the thermal cycling. Helicase dependent amplification (HDA) mimics the in vivo DNA duplication scheme which uses the helicase to unwind the double stranded DNA (Jeong, Y. J., Park, K. & Kim, D. E. Isothermal DNA amplification in vitro: the helicase-dependent amplification system. Cellular and Molecular Life Sciences 66, 3325-3336 (2009); Gill, P. & Ghaemi, A. Nucleic acid isothermal amplification technologies—A review. Nucleosides Nucleotides & Nucleic Acids 27, 224-243 (2008); Vincent, M., Xu, Y. & Kong, H. M. Helicase-dependent isothermal DNA amplification. Embo Reports 5, 795-800 (2004)). Unlike other isothermal amplification approaches, HDA utilizes a much simpler reaction mechanism and exponentially amplifies the target sequence to a detectable level in a reasonably short time period. Compared to PCR, although HDA lacks sensitivity, the simple thermal management of HDA renders it useful for point-of-care applications.

One of the preconditions for nucleic acid amplification is to extract the genomic contents from crude biosamples. This sample preparation step removes inhibitors that may have negative effects on the amplification reactions. Conventional ethanol-precipitation based extraction methods require centrifugation and hence are not compatible with a chip format. Solid phase extraction, which works by promoting nucleic-acid adsorption in a chaotropic environment and desportion in low ionic strength buffers often employs a silica based substrate in the form of either micro-pillars or micro-posts (Cady, N. C., Stelick, S. & Batt, C. A. Nucleic acid purification using microfabricated silicon structures. Biosensors & Bioelectronics 19, 59-66 (2003); West, J., Boerlin, M., Jadhav, A. D. & Clancy, E. Silicon microstructure arrays for DNA extraction by solid phase sample contacting at high flow rates. Sensors and Actuators B-Chemical 126, 664-671 (2007)) or immobilized micro- or nano-particles (Pipper, J., Inoue, M., Ng, L. F. P., Neuzil, P., Zhang, Y. & Novak, L. Catching bird flu in a droplet. Nat. Med. 13, 1259-1263 (2007); Pipper, J., Zhang, Y., Neuzil, P. & Hsieh, T. M. Clockwork PCR including sample preparation. Angew. Chem. 47, 3900-3904 (2008); Gijs, M. A. M. Magnetic bead handling on-chip: new opportunities for analytical applications. Microfluidics and Nanofluidics 1, 22-40 (2004); Breadmore, M. C., Wolfe, K. A., Arcibal, I. G., Leung, W. K., Dickson, D., Giordano, B. C., Power, M. E., Ferrance, J. P., Feldman, S. H., Norris, P. M. & Landers, J. P. Microchip-based purification of DNA from biological samples. Analytical Chemistry 75, 1880-1886 (2003)) and therefore is easy to be adopted for microfluidic platforms. Yet to integrate the solid phase sample preparation into a μTAS system remains challenging; multiple procedures and reagents are involved in the sample preparation process, and fairly complex microfluidic architectures and peripherals are required. In addition, the typical microfluidic chip requires fluidic coupling for the introduction of external reagents and the release of processed waste. This traditional design is prone to cross contamination and inefficient transfer and utilization of reagents due to dead-volume associated with the fluidic coupling.

There thus remains a need for improved systems and components for biochemical assays.

### SUMMARY

A fluidic cartridge for biochemical assays according to an embodiment of the current invention includes a cartridge body defining a first droplet region and a second droplet region with a droplet restraining barrier therebetween. The droplet restraining barrier has a gap between the first and the second droplet regions. The fluidic cartridge also includes a first droplet dispensed in the first droplet region. The first droplet includes a plurality of magnetic particles dispersed therein. The fluidic cartridge also includes a second droplet disposed in the second droplet region. The plurality of magnetic particles are sufficiently small to be drawn through the gap between the first and second droplet regions when compelled by an applied magnetic field, and the first droplet is restrained by the restraining barrier while the plurality of magnetic particles are drawn through the gap.

A biochemical assay system according to an embodiment of the current invention includes a stage adapted to receive a fluidic cartridge, and a magnetic control assembly that includes a magnet. The magnet of the magnetic control assembly is movable to direct motion of magnetic particles contained within the fluidic cartridge.

### BRIEF DESCRIPTION OF THE DRAWINGS

Further objectives and advantages will become apparent from a consideration of the description, drawings, and examples.

FIGS. 1A and 1B are schematic illustrations of embodiments of fluidic cartridges, microfluidic chips in these examples, that are primed with buffer droplets. Devices are fabricated by casting polydimethylsiloxane (PDMS) against molds. All buffer droplets are dispensed to the designated position as illustrated in the figures. In FIG. 1A the device was fabricated using a lithographically patterned mold. This particular design allows three samples to be processed in parallel. All droplets sit in air medium in this example. In FIG. 1B the device is fabricated using a CNC machined mold with a mini oil tank. Droplets all sit in the oil medium. In both cases, samples are first mixed with lysis/binding buffer and SSP. The SSP moves from Lysis/Binding buffer droplet to washing buffer 1, washing buffer 2, second washing buffer 2 followed by the PCR/elution buffer. At the end, the SSP drags the PCR/elution buffer to the micro reaction basin where the amplification reaction takes place.

FIG. 2 provides a demonstration of surface topography assisted droplet manipulation according to a couple of embodiments of the current invention. From right to the left, the SSP first is mixed with the droplet on the right. The permanent magnet actuates the SSP which in turn drags the droplet along to the aperture (also referred to as a gap). The droplet is blocked by micro-elevations but the SSP can squeeze through the aperture. After splitting from the first droplet, the SSP plug moves towards and merges with the droplet on the left. Two series of pictures demonstrate the surface topography assisted droplet manipulation in a) (top) in air as a medium and b) (bottom) in an oil medium, respectively.

FIGS. 3A and 3B are schematic illustrations of embodiments of fluidic cartridges according to further embodiments of the current invention. In both examples, all required reagents are stored on chip in the form of droplet. FIG. 3A: In one example (a), the entire chip is sealed with a removable cover film that is detached before usage. FIG. 3B: In another example (b), the entire chip is permanently bonded with a plastic or glass plate that comprises a small opening designed for sample introduction. The open area is sealed with a removable cover film that can be detached to allow for the introduction of a sample droplet.

FIGS. 4A and 4B provide schematic illustrations of microfluidic chips primed with buffer droplets for cell lysis, DNA extraction, purification and amplification according to a couple of embodiments of the current invention. All buffer droplets were dispensed at the designated positions. FIG. 4A is a device design that used V-shaped slits to assist SSP splitting. All droplets sat in air except the reaction buffer droplet which was covered by mineral oil. FIG. 4B is a device design that employed pairs of micro pillars to facilitate SSP splitting. The mini tank was filled with mineral oil, and all droplets sat in the oil medium. For sample preparation, the SSP moved from the lysis/binding buffer droplet (404 in FIG. 4A) to washing buffer 1 (406), washing buffer 2a (408), washing buffer 2b (410), and ended at the amplification reaction buffer droplet (412). Similar droplets were used in FIG. 4B. Finally, the SSP dragged the reaction buffer droplet to the micro reaction basin where the amplification reaction took place.

FIGS. 5A and 5B provide a schematic illustration of a fluidic cartridge according to another embodiment of the current invention. FIG. 5B, in addition to surface topographical features, electrodes are also incorporated to aliquot droplets using electrowetting on dielectric (EWOD).

FIGS. 6A-6D illustrate embodiments of microfluidic manipulation and biochemical assay systems according to some embodiments of the current invention.

FIG. 7 illustrates an embodiment of microfluidic manipulation and biochemical assay system that includes a fluorescence detection system.

FIG. 8 illustrates an embodiment of a fluorescence detection system that can be used with biochemical assay systems according to an embodiment of the current invention.

FIG. 9 is a schematic of a fluorescence detection circuit that can be used with the fluorescence detection system of FIGS. 7 and/or 8. The fluorescence detection module applies a 90-degree excitation-emission arrangement to minimize the optical noise. An LED serves as the excitation source. A software timer controls ON/OFF of the LED. During its ON time, the LED blinks at 500 Hz modulated by the square pulse generated by the driver circuit. The emission signals collected by the photodiode are filtered and amplified by a lock-in amplifier modulated to 500±50 Hz. The phase sensitive configuration allows the optical system to operate in ambient light environment, which is important for POC applications.

FIG. 10 is a schematic illustration of a method of producing fluidic cartridges according to an embodiment of the current invention.

FIGS. 11A-11D provide data that show comparisons of critical volumes on a flat surface and on a surface with topographical features for a) water, b) 100% isopropanol (IPA), c) 70% ethanol and d) mineral oil (Sigma Aldrich M5904) droplet at different SSP amount.

FIG. 12 shows results for gDNA extraction in droplets on the presented device validated in a separate experiment where the extracted gDNA is collected and run on a 0.8% agarose gel with the HindIII digested λDNA as marker at 8V/cm for 90 mins according to an embodiment of the current invention.

FIGS. 13A and 13B show data for Rsf-1 biomarker detection using real time PCR according to an embodiment of the current invention. FIG. 13A shows the real time PCR amplification curve. FIG. 13B shows melting curve analysis of the amplicon.

FIGS. 14A and 14B show data for Rsf-1 biomarker detection using real time HDA according to an embodiment of the current invention. FIG. 14A shows the real time HDA amplification curve. FIG. 14B shows melting curve analysis of the amplicon.

FIG. 15 shows E. coli identification using real time PCR according to an embodiment of the current invention. The TaqMan probe targets a specific sequence that is unique to the E. coli.

FIG. 16 is a schematic illustration of a biochemical assay system according to an embodiment of the current invention.

FIG. 17 is to explain data analysis software for high resolution melting curve analysis according to an embodiment of the current invention. Left: the raw data. Middle: Each curve is normalized and smoothened. A hierarchical clustering algorithm is used to group similar curves. Right: The software can also perform difference curve analysis with automatic or user defined reference.

FIGS. 18A-18C show normalized melting curves for test samples according to an embodiment of the current invention. (FIG. 18A) Kras: 30 bp synthetic segment of two variants (wildtype and G>A mutant) were used. (FIG. 18B) Beta-globin: 110 bp synthetic segment of three variants were used, 1) wildtype; 2) single mutant (c.20A>T); 3) double mutant (c.20A>T; c.9C>T). (FIG. 18C) CDKN2B (p15) promoter: Two variants of 60 bp synthetic segment were tested using simulated products of a bisulfite-converted PCR: 1) wildtype promoter (unmethylated); 2) methylated promoter. Melt curve profiles for Kras show clear separation between homozygous wildtype and mutant heterozygote, marked by higher melting temperature of the wildtype. HBB profiles show a clear separation between three variants, with double mutation heterozygote clearly marked by double transition. p15 promoter melt curve profiles also show clear difference based on methylation status.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

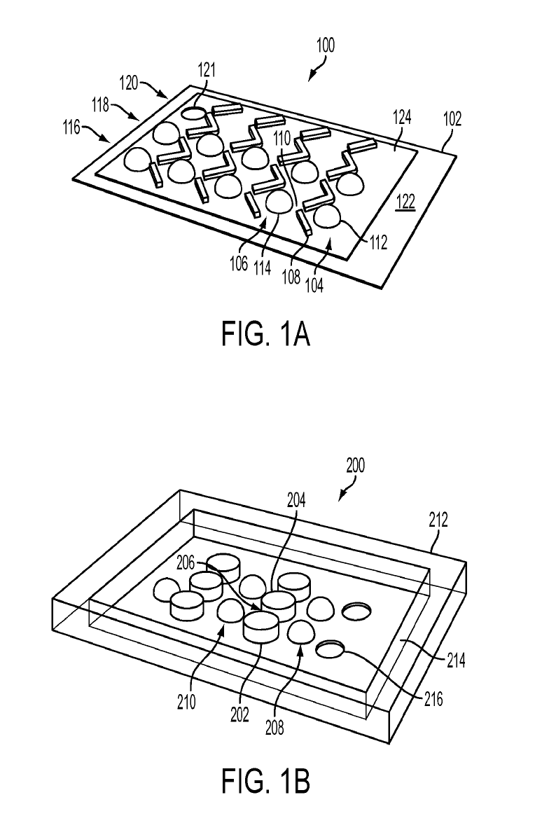

According to an embodiment of the current invention, we have developed a droplet microfluidic platform that is able to perform nucleic-acid based pathogen identification and biomarker detection from crude bio-samples. In one embodiment, we utilize silica superparamagnetic particles (SSP) to carry out solid phase extraction in discrete droplets. Material transfers are realized by moving, mixing, merging and splitting the droplets. The SSP provide both the actuation force for the droplet movement and the functional substrate for DNA attachment. The fluidic cartridge, which can be a microfluidic chip, has a unique surface with various topographical features that facilitate the droplet manipulation. For example, micro-surface elevations form an aperture to provide large surface tension and friction, allowing the SSP to split from the droplet (Zhang, Y. & Wang, T. H. Geomorphology-assisted manipulation of magnet-actuated droplet for solid phase DNA extraction and droplet-in-oil PCR. IEEE MEMS 2010 conference proceeding (2010); Shikida, M., Takayanagi, K., Honda, H., Ito, H. & Sato, K. Development of an enzymatic reaction device using magnetic bead-cluster handling. Journal of Micromechanics and Microengineering 16, 1875-1883 (2006)). According to some embodiments, micro-reaction basins hold the droplet in position during the amplification reaction, avoiding temperature induced droplet motion. In some embodiments, all of the reagents and buffers can be pre-stored in the form of droplets that are encapsulated by mineral oil, for example. Reagents and chemicals can be stored in the cartridge in the form of droplets. A fluidic cartridge according to an embodiment of the current invention can be sealed with a plastic film, forming a self-sustained cartridge for nucleic acid-based biological detection. A system for biochemical assays according to an embodiment of the current invention can include a sample handling stage that is designed to integrate the fluidic cartridge, a magnet bar holder and thermal cycling unit onto a single platform. According to some embodiments, the stage can be disassembled for easy transportation and reassembled on site. We have constructed and tested a platform according to an embodiment of the current invention by successfully identifying E. coli and detecting Rsf-1 cancer biomarkers from crude bio-samples using real time PCR and HDA. FIG. 1A is a schematic illustration of a fluidic cartridge 100 for biochemical assays according to an embodiment of the current invention. The fluidic cartridge 100 includes a cartridge body 102 defining a first droplet region 104 and a second droplet region 106 with a droplet restraining barrier 108 between them. The droplet restraining barrier 108 has a gap 110 between the first droplet region 104 and the second droplet region 106. A first droplet 112 is dispensed in the first droplet region 104. The first droplet 112 has a plurality of magnetic particles dispersed in it. The size of droplets can range from sub-microliter to hundreds of microliter, for example. A second droplet 114 is disposed in the second droplet region 106. Each magnetic particle of the plurality of magnetic particles is sufficiently small so it can be drawn through the gap between the first and second droplet regions when compelled to so move by an applied magnetic field. The magnetic particles can have sizes ranging from nanometers to micrometers, for example. The surface can be unfunctionalized or functionalized with various surface chemistry. The first droplet 112 is restrained by the restraining barrier 108 while the plurality of magnetic particles is drawn through the gap 110. Fluidic cartridges according to embodiments of the current invention can have two or more droplets. The general concepts of the current invention are not limited to the particular number of droplets. For example, the fluidic cartridge 100 is an example of an embodiment of five droplets in a linear pattern 116 with a constraining barrier or corresponding gaps between each corresponding pair of droplets.

The fluidic cartridge 100 also has a second linear pattern 118 of five droplets with a restraining barrier between each corresponding pair of droplets. The fluidic cartridge 100 is also structured to have a third linear pattern 120 of five droplets with a restraining barrier between each corresponding pair of droplets. In the third linear pattern 120, the droplets are not shown to allow a view of the corresponding droplet regions. The fluidic cartridge 100 is an example in which three parallel sets of biochemical processes can be conducted. Fluidic cartridges according to other embodiments of the current invention can have one, two, three or more parallel reaction paths, as desired. Furthermore, the reaction pathways between adjacent droplets do not have to be in a linear arrangement as illustrated in the example of FIG. 1A. One or more structures can be included in the fluidic cartridge 100 to help stabilize droplets in certain positions, as desired by the particular application. For example, a well-like depression, such as depression 121, can be included in the fifth droplet position for each pathway 116, 118 and 120. This can be useful to help maintain the droplet in the fifth position as a reaction position, such as for a PCR reaction in one embodiment. Since the PCR reaction uses thermal cycling, a depression such as 121 can be useful to help maintain the droplet in the desired position during the thermal cycling, for example.

The cartridge body 102 can include a substrate 122 with micro-patterned layer 124, for example, that can also include restraining barriers, such as restraining barrier 108. However, the general concepts of the current invention are not limited to cartridges constructed with a micro-patterned layer attached to a substrate. Although not shown in FIG. 1A in order to show internal structures, the fluidic cartridge 100 can also include an enclosing structure to provide a self-contained fluidic cartridge for storage, distribution and use. In addition, some or all inner surfaces of the fluidic cartridge 100 can be treated or coated with a material to provide desired surface properties, for example to encourage the formation of droplets. Wetting properties on inner surfaces of the fluidic cartridge 100 can be designed as desired for the particular application.

FIG. 1B is a schematic illustration of a fluidic cartridge 200 for biochemical assays according to another embodiment of the current invention. Many features of the fluidic cartridge 200 are similar to that of the fluidic cartridge 100; however, in this embodiment, the restraining barriers are formed by adjacent cylindrical formations, such as cylindrical formations 202 and 204 spaced to reserve a gap 206 between first droplet region 208 and second droplet region 210. The fluidic cartridge 200 is an example in which the processing path is more complicated than a single straight line. This will be described in more detail below. In addition, an enclosing structure 212 is schematically illustrated in FIG. 1B. Fluidic cartridges according to some embodiments of the current invention can also include a fluid, such as a liquid, in which the droplets are immersed. For example, the fluidic cartridge 200 can include a fluid 214 in which the droplets are immersed. The fluid 214 can be, but is not limited to, a mineral oil. Also illustrated in FIG. 1B is a reaction region 216.

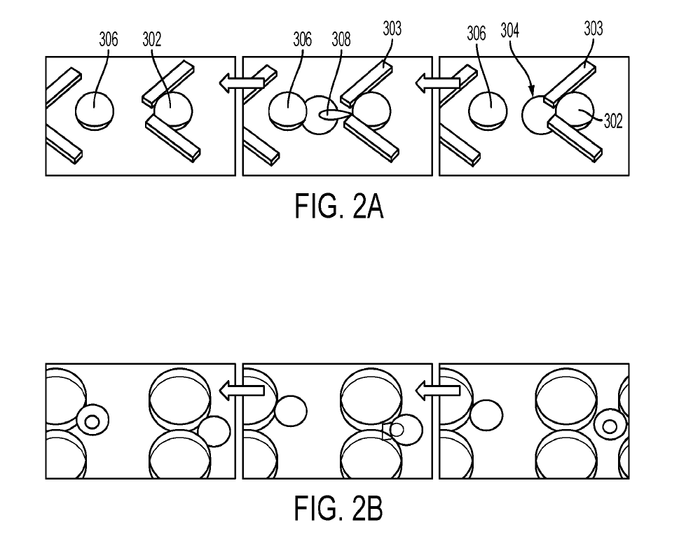

FIG. 2 shows a sequence of images to illustrate the operation of fluidic cartridges according to some embodiments of the current invention. The upper sequence is for a fluidic cartridge that has wedge-shaped restraining barriers, similar to those illustrated in FIG. 1A. The lower sequence is for a fluidic cartridge that has cylindrical-shaped restraining barriers, similar to those illustrated in FIG. 1B. In the top right-hand image, the droplet 302 has been drawn to the restraining barrier 303 by magnetic particles dispersed in it. The magnetic particles are beginning to be drawn through the gap 304 towards the droplet 306. In the center, upper figure, the magnetic particles 308 have been separated from the droplet 302 while the droplet 302 is being restrained by the restraining barrier, for example by friction and surface tension. The magnetic particles and any molecules or other material attached to them are in the form of a plug 308. In this example, a magnet is arranged below droplet 302 in the right-hand image and moved towards droplet 306 such that the plug 308 merges with droplet 306 in the far left-hand image in the three-image sequence. The lower sequence of three images in FIG. 2 is similar to the upper sequence except for the cylindrical restraining structures.

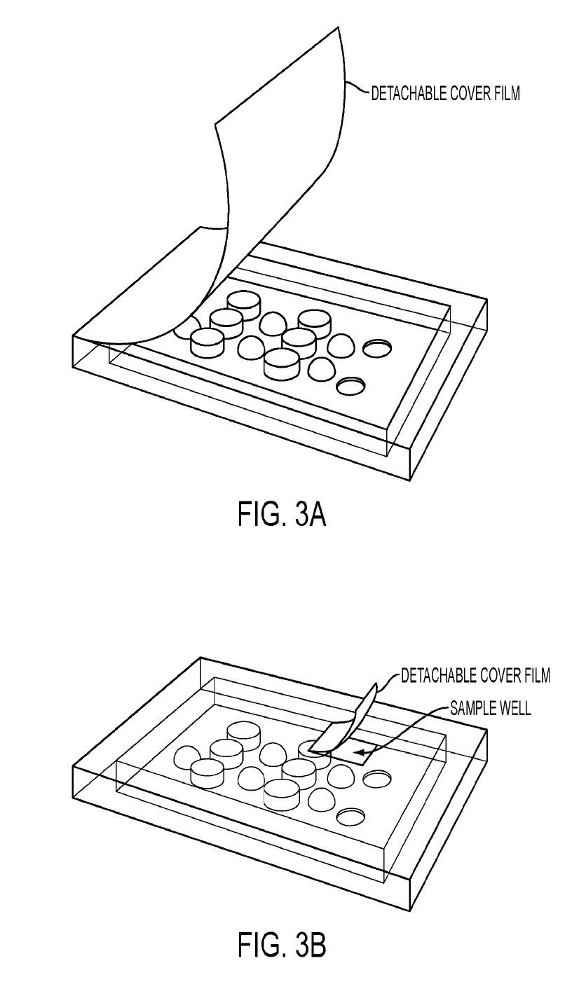

FIGS. 3A and 3B illustrate two embodiments of fluidic cartridges that include detachable cover films. The detachable cover films can be attached during the manufacture of the fluidic cartridge such that it can be shipped and stored preloaded for use. The detachable cover films are peeled open so that one or more samples can be introduced into the fluidic cartridge during use. In general, further materials can be added during processing, if desired. However, some embodiments of the fluidic cartridges can be self-contained except for the particular samples to be added at the time of use. In FIG. 3A, there is a large detachable cover film over most of, or the entire, top surface of the fluidic cartridge. This can be useful if multiple samples and/or other material will be added at the time of use. In FIG. 3B, there is a detachable cover film over an individual sample well. The general concepts of fluidic cartridges according to the current invention are not limited to only these examples. One can imagine a broad range of packaging structures that are possible for fluidic cartridges according to various embodiments of the current invention.

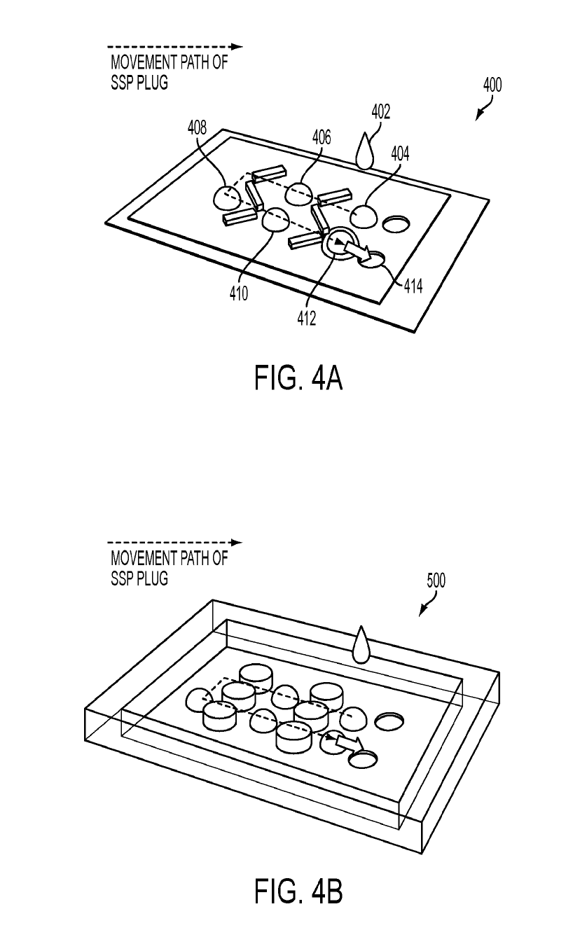

FIG. 4A illustrates a fluidic cartridge 400 according to another embodiment of the current invention. In this example, a sample droplet 402 is introduced into the fluidic cartridge 400 for processing. At least a portion of the sample droplet 402 is mixed with the droplet 404, which contains magnetic particles dispersed in it. After the initial processing of the sample in the droplet 402, at least some of the magnetic particles are drawn through the gap in a restraining barrier by an applied magnetic field to merge with droplet 406 (see dashed line). The magnetic particles take material attached to them along with them to the droplet 406. The magnetic particles are then separated from droplet 406 by another restraining barrier that has a gap through it, to reach droplet 408, as indicated by the dashed line. In this step, it can be the plug of magnetic particles that change direction in motion, or the plug of magnetic particles can merge with the droplet 408, which is then moved as a droplet due to the magnetic particles merged with it. The process continues by the separation of magnetic particles from the droplet 408, merging with droplet 410, separating from droplet 410 and merging with droplet 412, and finally moving droplet 412 to a reaction well 414. FIG. 4B illustrates a fluidic cartridge 500 according to another embodiment of the current invention. The fluidic cartridge 500 is similar to fluidic cartridge 400 except that cylindrical structures are provided to form the restraining barriers providing a gap for the magnetic particles.

In some embodiments, the droplet 404 can be a lysing and binding buffer solution, for example to lyse cells and bind DNA to the magnetic particles. The droplet 406 can be a washing buffer solution, for example. The droplet 408 can be, for example, a second washing buffer solution which can be the same as or different from that of droplet 406. The droplet 410 can be, for example, a third washing buffer solution which can be the same as or different from that of droplet 408. Finally, the droplet 412 can be, for example, elution/reaction buffer solution. For example, the elution/reaction buffer solution can be, but is not limited to, a solution for performing a DNA amplification reaction, such PCR or HDA.

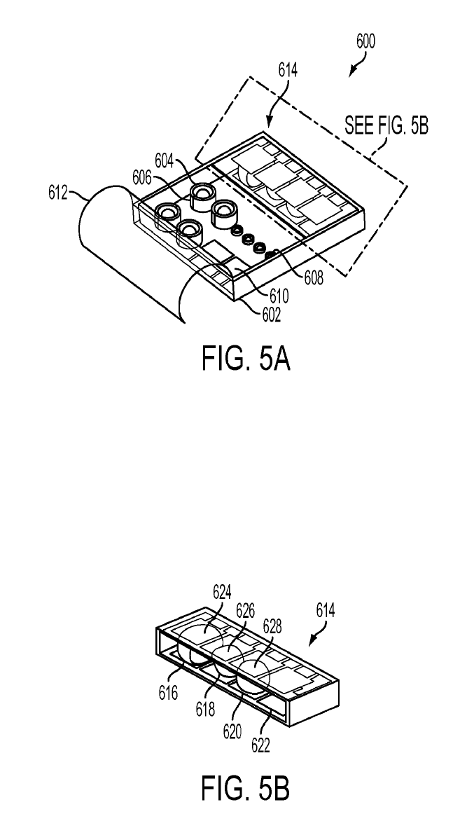

FIG. 5A illustrates a fluidic cartridge 600 according to another embodiment of the current invention. The cartridge 600 has a cartridge body 602 that defines a plurality of droplet restraining barriers, such as droplet restraining barrier 604. Each of the droplet restraining barriers has one or more gaps, such as gap 606, that allow magnetic particles to pass through while restraining the droplet due to friction and surface tension. Restraining barriers in this embodiment are in the form of a collar to help maintain the droplets in position during shipping and storage, for example. The fluidic cartridge also shows an example of magnetic material, such as magnetic component 608, which can be embedded into the fluidic cartridge to help hold magnetic particles in a stable position until overcome by a stronger magnetic force. For example, a magnetic component, such as magnetic component 608, can be arranged under a droplet held within restraining barrier 604. This can be useful for preventing magnetic particles from dispersing out through the gaps, such as gap 606, during shipping, storage, etc. prior to use.

The fluidic cartridge 600 can also have heater elements, such as heating element 610 arranged under a droplet position. In FIG. 5A, the processing pathways are linear in which there are two droplets. Some of the structures of the fluidic cartridge 600 are not shown for clarity. A heating element similar to heating element 610 can be arranged under one or more of the plurality of reaction droplets in the fluidic cartridge 600. The heating elements can be useful for performing PCR, for example. However, heating elements can be included other embodiments for other purposes than performing PCR. The fluidic cartridge 600 further includes a detachable cover film 612. The fluidic cartridge 600 can also be filled with a fluid, such as, but not limited to, mineral oil. In addition, the fluidic cartridge includes a sample splitting section 614. The sample splitting section 614 is shown in more detail in a cut-away view in FIG. 5B. The sample splitting section 614 includes a plurality of electrodes to provide additional control and manipulation of droplets. For example, the electrodes 616, 618, 620 and 622 can control wetting properties along the splitting section 614 by electrowetting-on-dielectrics (EWOD) for splitting and/or merging droplets. In the example of FIG. 5B, a sample droplet 624 is in the process of being split into a plurality of droplets, i.e., droplets 626 and 628 in FIG. 5B. In one example, the extracted DNA is eluted directly in the amplification (e.g. PCR) reagent and split into multiple daughter droplets using EWOD; hence multiple reactions are possible with a single sample preparation. This embodiment can be important for applications such as real-time quantitative PCR that requires triplicate/multiplicate analysis. In another example, the extracted DNA is eluted using elution buffer and is split into multiple daughter droplets. Each of the daughter droplets is moved and merged to another droplet of amplification reagent containing a specific primer. Thus, multiplex analysis of multiple sequences or genes can be achieved.

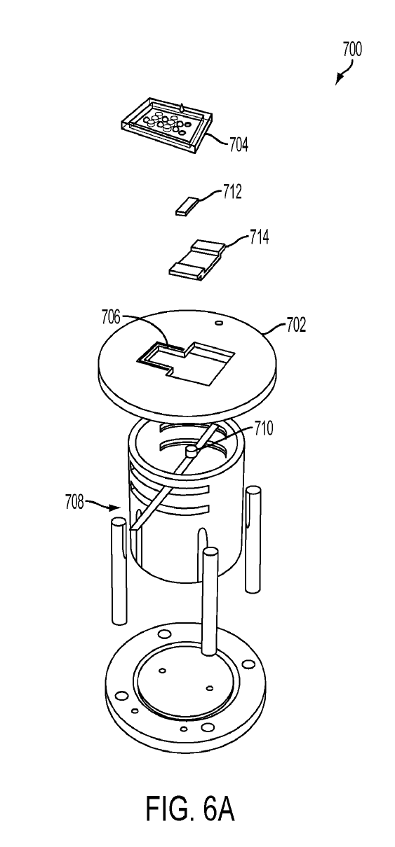

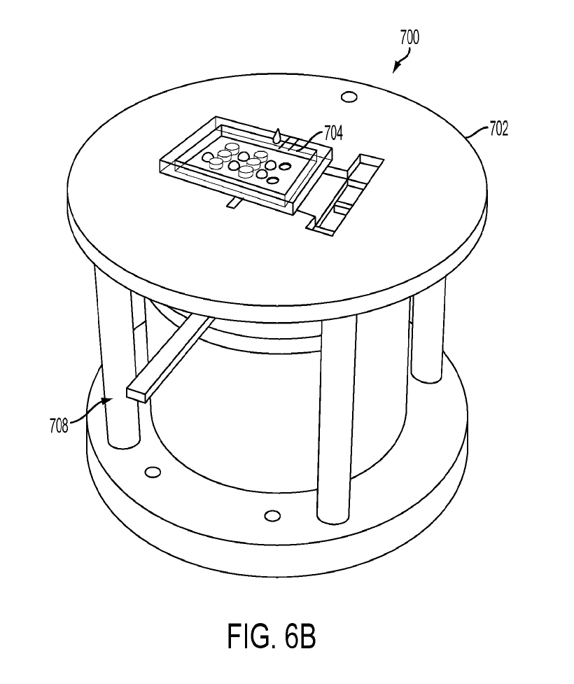

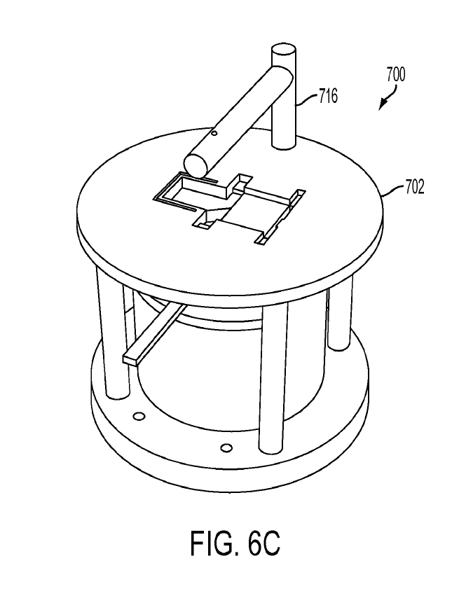

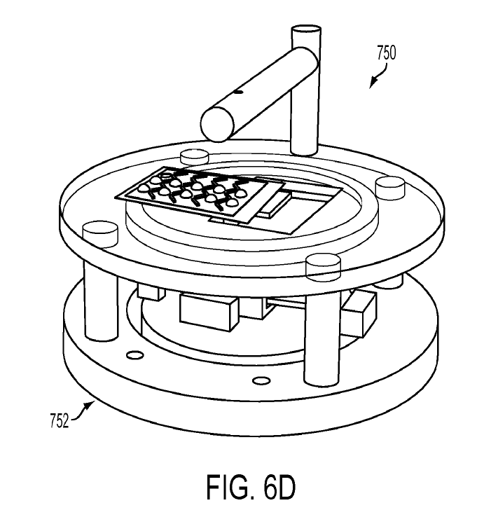

FIG. 6A shows an exploded view of a biochemical assay system 700 according to an embodiment of the current invention. FIG. 6B shows the biochemical assay system 700 in an assembled view. The biochemical assay system 700 includes a stage 702 adapted to receive a fluidic cartridge 704. In this embodiment, there is a vacuum trench 706 defined by the stage 702. The biochemical assay system 700 also includes a magnetic control assembly 708 that includes a magnet 710. The magnet 710 can be a permanent magnet, as in this example; however, it could also be an electromagnet if desired. The magnet 710 of the magnetic control assembly 708 is movable to direct motion of magnetic particles contained within said fluidic cartridge 704. The biochemical assay system 700 can also include a Peltier heater/cooler 712 to selectively heat and/or cool portions of the fluidic cartridge 704. A slider 714 can also be included which can be used so that the Peltier heater/cooler can movable. FIG. 6C shows an embodiment of the biochemical assay system 700 that includes a cross bar 716 that can be used to hold instruments, sensors, etc. such as a thermocouple, for example. The biochemical assay system 700 can be compact, such as a hand-held device, according to some embodiments of the current invention. It can also be constructed so it can be easier assembled, disassembled and re-assemble in some embodiments of the current invention.

FIG. 6D shows an embodiment of a biochemical assay system 750 according to another embodiment of the current invention. The biochemical assay system 750 can be similar to biochemical assay system 700, except magnetic control assembly 752 is automated with motorized translation stages rather than manually operated as in the biochemical assay system 700.

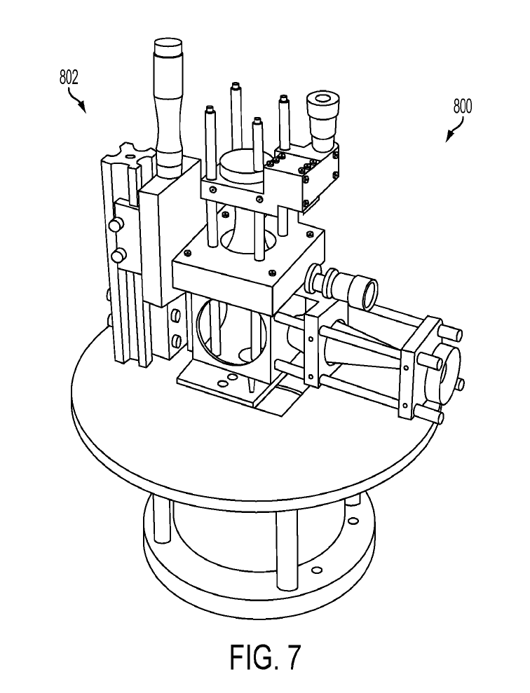

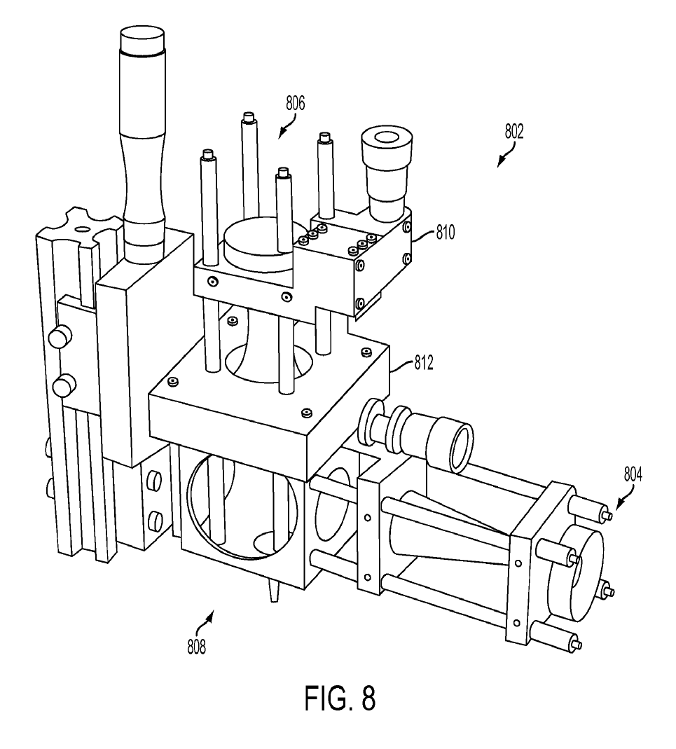

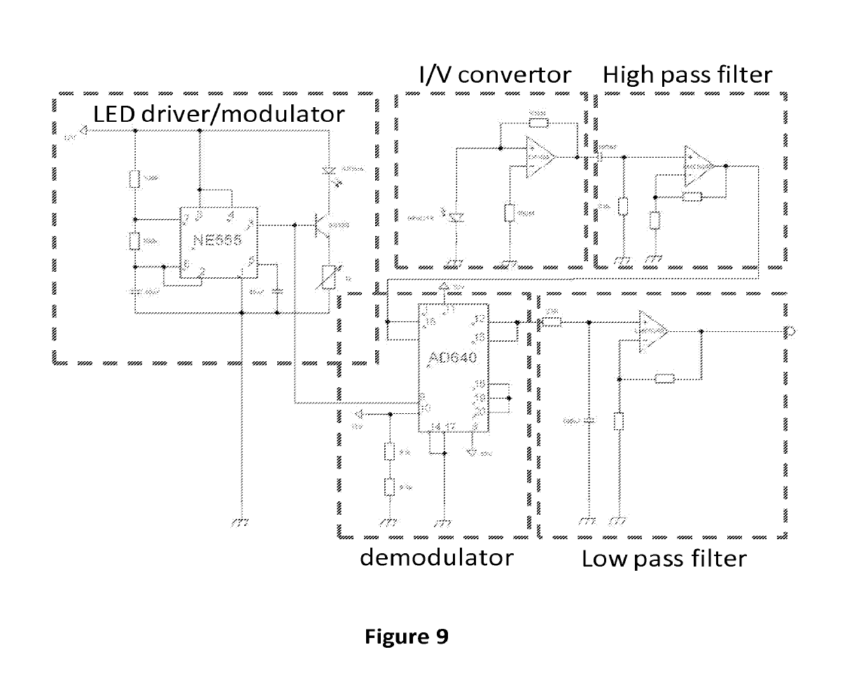

FIG. 7 shows an embodiment of a biochemical assay system 800 according to another embodiment of the current invention. The biochemical assay system 800 has a fluorescence detection system 802. In some embodiments, the fluorescence detection system 802 can be detachable such as is shown in FIG. 8. In this example, the fluorescence detection system is attached to the structure illustrated in FIG. 6B. The fluorescence detection system 802 includes an illumination system 804 and a detection system 806. The illumination system 804 includes a light source such as at least one light emitting diode (LED) and/or laser, for example. The illumination system 804 can also include optical filters to selectively filter the illumination light. The detection system 806 can include one or more photodiodes, for example. The fluorescence detection system 802 can also include an imaging optical system that includes an objective lens system 808 and a focusing lens system 810. Each of the objective and focusing lens systems can be single and/or compound lenses, depending on the particular application. In addition, refractive, diffractive and/or gradient index lenses can be used according to the particular application. The fluorescence detection system 802 can also include one or more emission filters, such as emission filter 812. FIG. 9 provides schematic of the fluorescence detection circuit for the fluorescence detection system 802 according to an embodiment of the current invention.

### EXAMPLES

### Surface Topography Assisted Droplet Manipulation

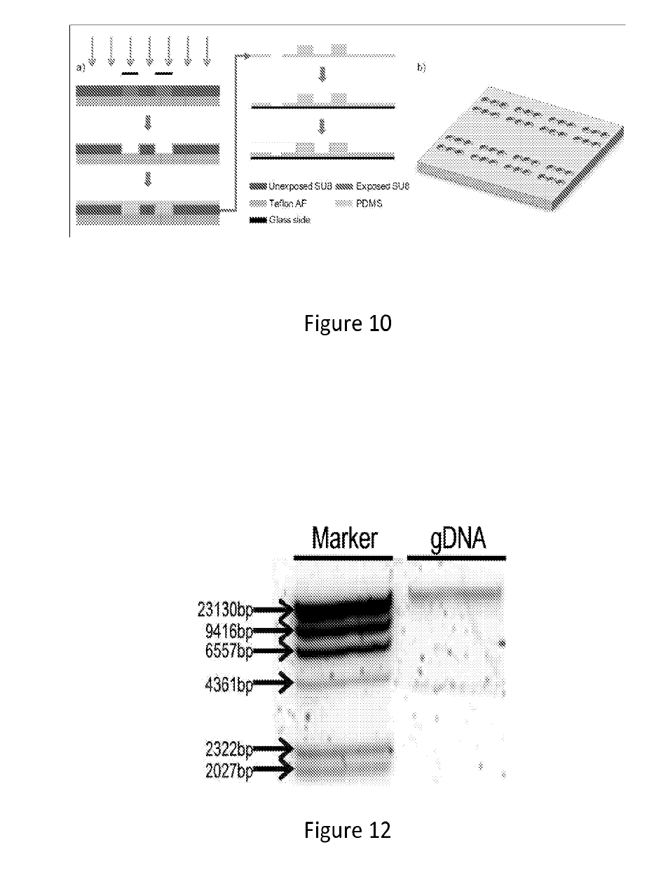

The desired surface features were made by casting polydimethylsiloxane (PDMS) against a micro-fabricated or micro-machined mold. For the micro-fabricated mold, a layer of 600 μm of SU-8 photoresist was spun on the silicon substrate by multiple spin coatings, and the patterns were lithographically defined (FIG. 10). The SU-8 mold was hard baked and dipped coated with 1% Teflon AF (Dupont Corp.) before PDMS casting. The PDMS was spun on the mold at 100 rpm for 30 s, resulting in a thin PDMS membrane. Micro reaction basins were created by punching through the membrane using a hollow puncher of Ø4 mm. Lastly, the membrane was oxygen plasma treated and rolled onto the glass coverslip using a metal rolling pin. The oxygen plasma bonded device was baked at 80° C. overnight, after which it was dip-coated with 1% Teflon AF and baked at 80° C. for another 24 hrs. The Teflon coating on the PDMS membrane rendered the surface more hydrophobic and eased the droplet movement. The micro-machined mold was designed using the computer aided design (CAD) software SolidWorks (Solid Work Corp.) and created using computer numeric controlled (CNC) machining (FIG. 10). We chose Polytetrafluoroethylene (PTFE) as the substrate material to avoid the Telfon coating on the mold. The PDMS casting process on the micro-machined mold was the same as the micro-fabricated mold. In additional, an optional mini tank could be created by bonding a hollow PDMS trunk around the PDMS membrane to contain the mineral oil. The droplet movement took place either in the air medium (FIG. 1A) or in the oil medium (FIG. 1B).

When moving at a given speed on a plane surface, there exists a critical volume below which the SSP drags the droplet along rather than splits from it. With increased amount of SSP, the critical volume generally becomes larger (Long, Z., Shetty, A. M., Solomon, M. J. & Larson, R. G. Fundamentals of magnet-actuated droplet manipulation on an open hydrophobic surface. Lab on a Chip 9 (2009)). In this example, micro-elevations are paired up to form a narrow aperture (or gap) with 500 μm space in between. The aperture provides a narrow path for the SSP plug while the micro-elevations stop the droplet from passing through due to large surface tension and friction. During the operation, the droplet is pulled by the SSP plug which travels together with the permanent magnet below it until the droplet reaches the aperture (see, e.g., FIG. 2), where the SSP plug can easily split from the liquid droplet and pass through the aperture while the liquid droplet is stopped by the micro-elevations. To aid visualization, we demonstrate the droplet manipulation on the open surface with aqueous food dye. The droplet manipulation is demonstrated in both the air medium (top (a) of FIG. 2) and the oil medium (bottom (b) of FIG. 2). The ease of splitting the SSP from the droplet at the aperture in a controlled manner is clearly shown in these examples. Because of the assistance of the micro-elevations, the critical volume becomes very small (≦1 μL) and fairly consistent according to some embodiments of the current invention.

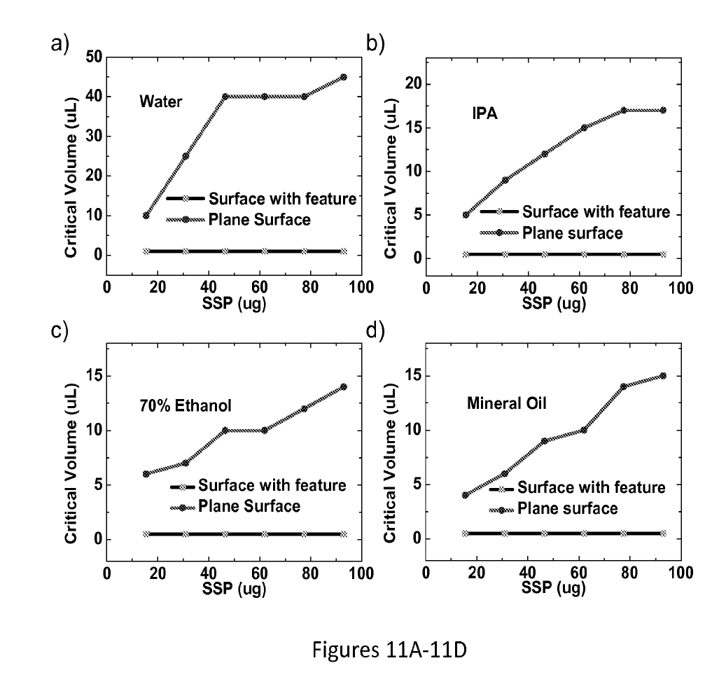

To compare the critical volume on a flat PDMS surface and some embodiments of the current invention, we estimated the critical volume by placing the permanent magnet ˜5 mm ahead of the droplet containing SSP. The measurements were carried out in air. Both the flat PDMS surface and our platform were dip-coated with Teflon. The liquid was slowly titrated to the droplet until the SSP could no longer drag but split from the droplet. On one hand, for all four buffers tested on the flat PDMS surface, including water, 100% IPA, 70% ethanol and mineral oil (M5904, Sigma-Aldrich), the critical volume became larger with the increasing amount of SSP (FIGS. 11A-11D). On the other hand, fairly small critical volume (≦14) was observed on our device regardless the amount of SSP used. The results demonstrate that the conditions for splitting the SSP from the liquid droplet with the assistance of the surface topographical features become less stringent, offering higher degree of flexibility and ease for manipulating droplets on an open surface. Whether the SSP plug moves jointly with the droplet or splits from the droplet is governed by the balance, or rather the imbalance, between the magnetic force exerted on the SSP and the surface tension/friction imposed on the droplet. In real world applications, various buffers with different surface energies are used at different volumes. In addition, many sample preparation processes involve droplet merging, which considerably alters the L/P ratio. If too much SSP is used, it is impossible to split the SSP from the droplet. If too little SSP is used, the magnetic force imposed on the SSP may not be enough to actuate the droplet. Furthermore, enough SSP must be applied to provide sufficient functional surface area for biomolecule adsorption. Hence, the range of SSP amount that can be used in a multi-procedure process is extremely narrow, and we must fine tune the SSP amount to accommodate the droplet manipulation. In contrast, with the assistance of the surface topographical features, a very wide range of SSP can be used since the SSP splitting becomes fairly easy. We can apply a relatively large amount of SSP for easy droplet actuation and efficient molecule catching without worrying about being able to split the SSP from the droplet.

### Reagent Pre-Stored Droplet Cartridge

Some embodiments of the current invention can be used for μTAS for point-of-care applications, for example. In the microfluidic cartridge, reagents and buffers can be pre-stored in the form of droplets, which are contained in the mineral oil. The mineral oil serves as insulation against evaporation. The droplets are confined by the surface topographical features, hence are prevented from moving inside the mineral oil and merging with other droplets. The device (see, e.g., FIGS. 3A and 3B) is sealed with adhesive sealing film or glass plate for the purpose of storage. Prior to use of the cartridge, the sealing film or plate is detached allowing for the user to introduce the biological sample to mix with the droplet containing lysis buffer and SSP.

### Multiplicate and Multiplex Analysis Through Droplet Splitting and Fusion

Some embodiments of fluidic cartridges according to the current invention can also have a micro-fabricated electrode array allowing for the generation and control of force fields such as electrowetting-on-dielectrics (EWOD) for splitting and merging droplets (see, e.g., FIGS. 5A and 5B). In one example, the extracted DNA is eluted directly in the amplification (e.g. PCR) reagent and split into multiple daughter droplets using EWOD; hence multiple reactions are possible with a single sample preparation. This design can be important for applications such as real-time quantitative PCR that requires triplicate/multiplicate analysis. In another example, the extracted DNA is eluted using elution buffer and is split into multiple daughter droplets. Each of the daughter droplets is moved and merged to another droplet of amplification reagent containing a specific primer. Thus, multiplex analysis of multiple sequences or genes can be achieved.

### Sample Handling Stage

The handheld sample handling stage according to an embodiment of the current invention shown FIGS. 7A-7C was designed using the SolidWorks and machined from the aluminium. CNC milling was applied when necessary. The stage comprised of a few parts including a T-bar, a slider, a top sample plate, four mounting poles, a center cylinder, a magnet bar holder and a base plate. The T-bar hung above the peltier heater and held the temperature sensor in position during the thermal cycling. It was constructed using the 0.5″ pole system from the Thorlab (Thorlab Inc.). The peltier was fixed onto the slider that slides beneath the micro reaction basins during nucleic acids amplification. The top sample plate had a cavity in the middle so that the magnets below the plate were able to reach the bottom surface of the chip and manipulate the droplet motion. A vacuum trench was created along the edge of the cavity. Once connected to the vacuum source from the backside of the top sample plate, it holds the chip steady. The magnet bar holder was made of aluminium with a piece of steel fused in the centre region. Hence, the magnets could be easily fixed on the bar without weakening the magnetic field. When operated in manual mode, the center cylinder was mounted and the magnet bar holder was inserted into the slots on the center cylinder. By moving the magnet bar holder along the slots, the magnets actuated the SSP that controlled the motion of the droplets. The sample stage could also operate in automatic mode by replacing the center cylinder with a 2D translation track (FIG. 6D).

A miniaturized fluorescence detection system can be provided to monitor the nucleic acids amplification reaction in real time according to some embodiments of the current invention. The fluorescence detection system can function as a standalone unit (FIG. 8) or be integrated to the sample handling stage as part of the μTAS (FIG. 7). The configuration of the system is illustrated in FIG. 8. The fluorescence detection module applies a 90-degree excitation-emission arrangement to minimize the optical noise. An LED serves as the excitation source. The excitation and emission filters can be easily changed to accommodate the fluorophore used. The emitted fluorescence is collected by the objective and filtered by the emission filter. The filtered light is focused onto the photodiode by a focusing lens. Due to the low fluorescence intensity and small current generated by the photodiode, a lock-in amplifier circuit is employed to boost the signal to noise ratio (FIG. 9). A software timer controls ON/OFF of the LED. During its ON time, the LED blinks at 500 Hz modulated by the square pulse generated by the driver circuit. The emission signals collected by the photodiode are filtered and amplified by a lock-in amplifier modulated to 500±50 Hz. The phase sensitive configuration allows the optical system to operate in ambient light environment, which is important for POC applications. This lock-in configuration renders the system insensitive to the ambient optical noise. The current generated by the photodiode is first converted to voltage through an I/V conversion circuit with 10⁸ V/A gain. The signal is then demodulated and filtered by a high pass and a low pass filter before being acquired using a LabView program. For the real time amplification reaction, the fluorescence signal was sampled at 1 Hz with 100 ms bin time. A melting curve analysis was performed by ramping the temperature at 0.1° C./s. The fluorescence signal was sampled at 2 Hz with 100 ms bin time. The negative first derivative was taken to determine the melting temperature.

Demonstration with Detection of Rsf-1 Gene in Blood

The Rsf-1 is a chromatin remodeling gene that is believed to be a promising biomarker for ovarian cancer diagnosis and prognosis. Ovarian cancer patients with Rsf-1 gene amplification have more severe conditions and shorter survival than those without (Shih, L. M., Sheu, J. J. C., Santillan, A., Nakayama, K., Yen, M. J., Bristow, R. E., Vang, R., Parmigiani, G., Kurman, K. E., Trope, C. G., Davidson, B. & Wang, T. L. Amplification of a chromatin remodeling gene, Rsf-1/HBXAP, in ovarian carcinoma. Clinical Cancer Research 11, 9161S-9161S (2005)).

Detecting the Rsf-1 biomarker started with a 5 μL human whole blood sample. The device was first primed with buffer droplets. Binding buffer, washing buffer and SSP were purchased from Qiagen and prepared according to the manufacturer's protocol. 5 mL of lysis/binding buffer was mixed with 5 mL of isopropanol alcohol (IPA), 0.5 mL of protease and 0.5 mL SSP. The mixture was dispensed onto the chip as a droplet that sat on the surface. One drop of 17.5 mL washing buffer 1 and two drops of 12.5 mL washing buffer 2 were dispensed at their designated locations on the chip (FIG. 4). A 5 mL droplet of nucleic acid amplification reaction mixture, which also functioned as the elution buffer, was dispensed onto the chip. If the chip was operated in air, mineral oil was applied over the reaction buffer droplet in order to prevent evaporation during heating. With all the buffer droplets in position, the device was primed and ready for sample processing.

The SSP was incubated with lysis/binding buffer for 10 minutes during which the cells were ruptured and the gDNA adsorbed to the SSP surface. After the incubation, the droplet moved along with the SSP which was actuated by the permanent magnet. When the droplet reached the aperture, the SSP split from the parent droplet and formed a small plug, which was then moved to the first washing buffer droplet. Sequentially, the SSP plug moved through all washing buffer droplets in the same fashion. The gDNA extraction process ended when the SSP was separated from the last washing buffer droplet. Thus far, the gDNA was highly concentrated on the SSP surface and ready for downstream analysis. The surface-adsorbed gDNA was eluted from the SSP in the PCR or HDA buffer, the ionic strength and the pH conditions of which favored the gDNA desorption. In order to validate the gDNA extraction, we performed a separate experiment in which the eluted gDNA was collected and run on a 0.8% agarose gel with the HindIII digested λDNA as marker at 8V/cm for 90 mins. The isolated gDNA appeared as a gel band>23 kbp (FIG. 12).

Amplification reactions took place in the micro reaction basin which could hold the droplet in position during the amplification reaction. Moreover, the bottom surface of the micro reaction basin is exposed to the glass coverslip which was closer to the peltier and had better thermal conductivity than the PDMS. The detection was first carried out with real time PCR. The fluorescence signal was continuously monitored as PCR progressed.

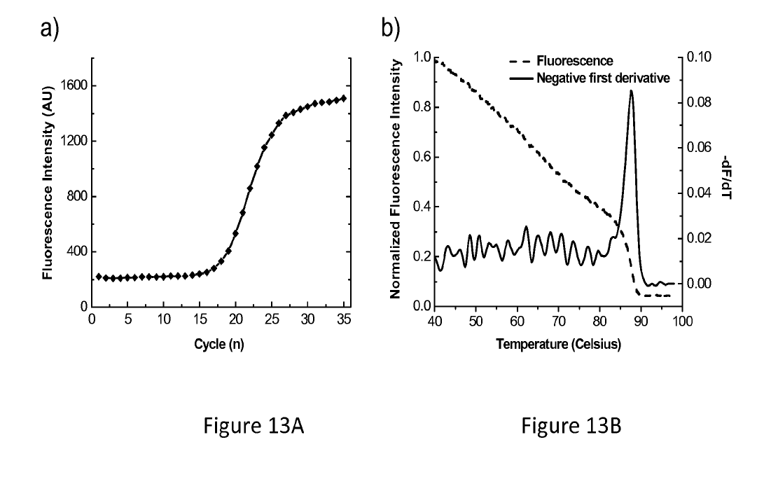

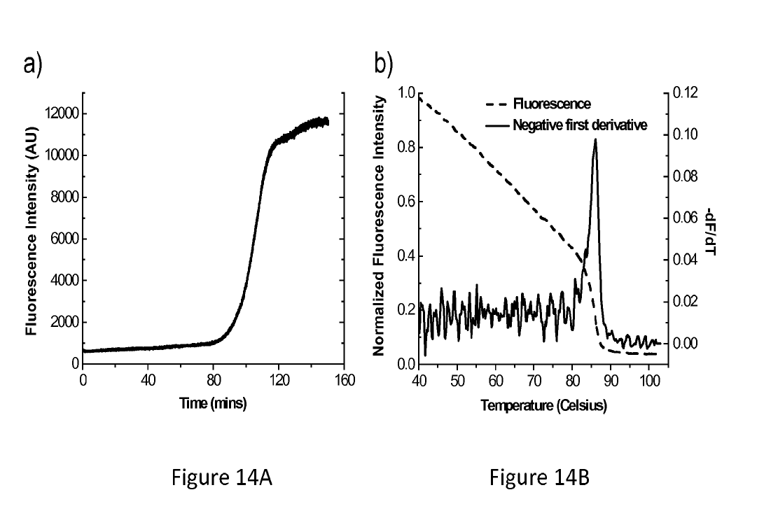

The successful detection of Rsf-1 gene from the blood sample was demonstrated by the real time PCR amplification curve (FIG. 13A). The specificity of the PCR product was evaluated by the melting curve analysis. A single peak observed in the negative first derivative graph confirmed the presence of a single amplicon (FIG. 13B). Rsf-1 detection was also carried out using HDA. Since no thermal cycling was involved, the fluorescence signal monotonically increased as the reaction proceeded. The Rsf-1 gene was exponentially amplified and successfully detected as shown in the real time HDA amplification curve (FIG. 14A). Again, the specificity of the amplicon was evaluated by the melting curve analysis and a single peak was observed (FIG. 14B).

Demonstration with E. Coli Detection

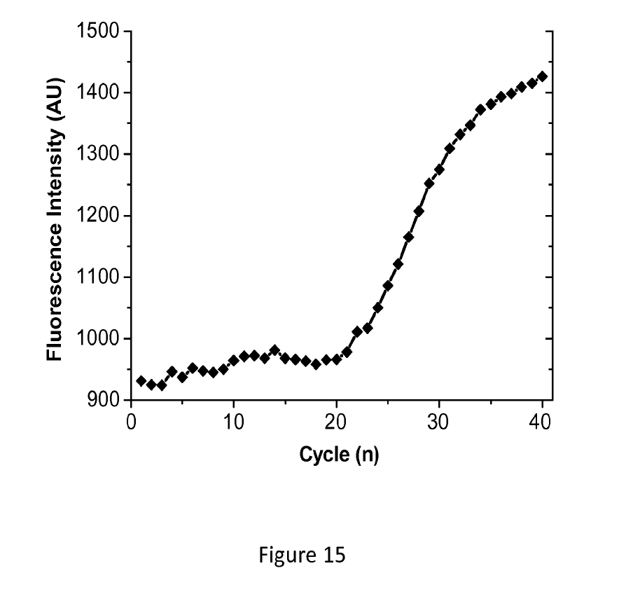

We used E. coli as our pathogen identification modeling system. The bacteria detection started with gDNA extraction followed by real time PCR with a previously validated sequence specific Taqman probe that identified E. coli (Yang, S., Ramachandran, P., Hardick, A., Hsieh, Y. H., Quianzon, C., Kuroki, M., Hardick, J., Kecojevic, A., Abeygunawardena, A., Zenilman, J., Melendez, J., Doshi, V., Gaydos, C. & Rothman, R. E. Rapid PCR-based diagnosis of septic arthritis by early gram-type classification and pathogen identification. Journal of Clinical Microbiology 46, 1386-1390 (2008); Yang, S., Lin, S., Kelen, G. D., Quinn, T. C., Dick, J. D., Gaydos, C. A. & Rothman, R. E. Quantitative multiprobe PCR assay for simultaneous detection and identification to species level of bacterial pathogens. Journal of Clinical Microbiology 40, 3449-3454 (2002)). The gDNA extraction was the same as that in the biomarker detection process. In addition, heat was applied during the cell lysis because the prokaryotic cells possessed a cell wall and were more resistant to lysing reagents. Since the Taqman probe could only be cleaved hence unquenced by the polymerase upon the binding to the template during the elongation, the increased fluorescence intensity suggested the successful amplification of the target sequence that was unique to the microbe species, which in this case was E. coli. The real time PCR amplification curve demonstrated successfully amplification and identification of E. coli (FIG. 15).

### Melting Curve Analysis

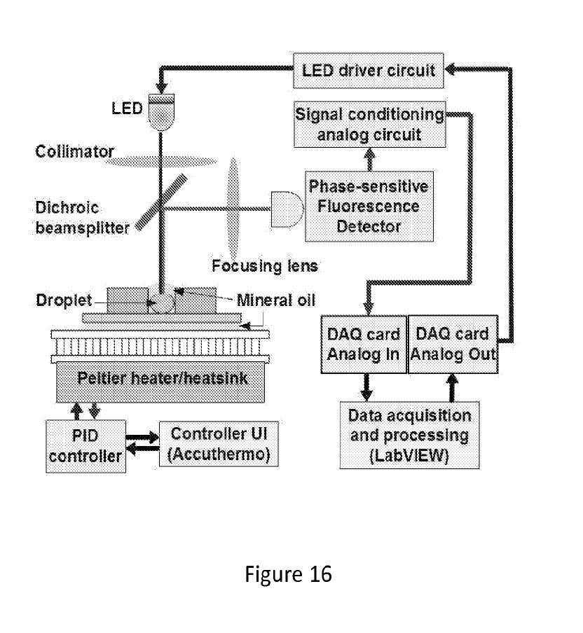

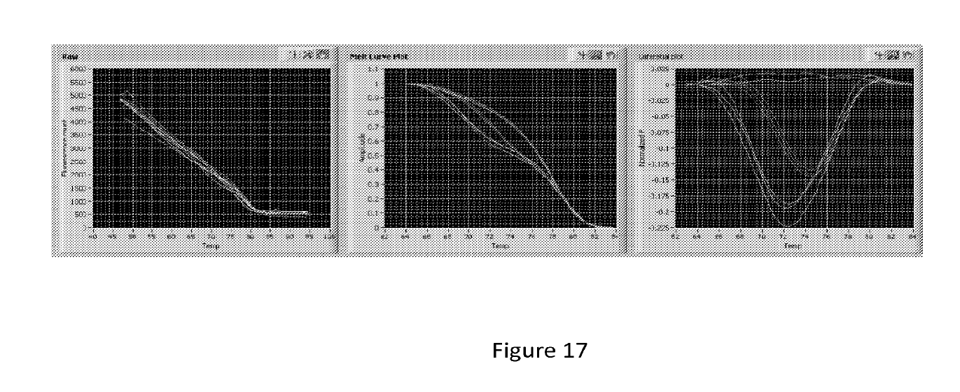

High resolution melting analysis is a simple yet powerful assay for rapid genotyping. Mutation site or other signature patterns are selectively amplified through PCR and the amplicons are stained with saturating intercalating dye such as LCGreen (Idoha) or EvenGreen (biotium). The temperature is then slowly ramped up with a typical resolution of 0.05-0.2° C./step while the fluorescent signals are continuously monitored. Due to the sequence difference, mutants and wildtype usually present distinctive melting curves. FIG. 16 is a schematic illustration of a biochemical assay system according to an embodiment of the current invention. This is an example of a portable system for genetic mutation detection and methylation analysis using a droplet-based microfluidic platform according to an embodiment of the current invention. Early screening of genetic markers for diseases enables targeted treatment and improved prognosis, making a strong case for point-of-care diagnostics. Using cancer biomarker KRAS codon 12 mutation and sickle-cell anemia biomarker rs334 polymorphism in human beta-globin gene (Krypuy M et al., BMC Cancer. 6:295 (2006); Herrmann M G et al., Clin Chem. 53:150-152 (2007)), this example demonstrates a portable implementation of droplet-based system for identifying genetic biomarkers of diseases. We also demonstrate identification of epigenetic cancer biomarker using CDKN2B promoter hypermethylation (Bailey V J et al., Genome Res. 19(8):1455-61 (2009)). DNA melting analysis was performed in a 10 μL droplet immersed in mineral oil. Measurement was performed between 50° C. and 95° C. with a resolution of 0.05° C./step. The sample temperature was calibrated to the temperature monitored from surface-mounted thermocouple proximal to the sample droplet. Fluorescence measurement was performed with 90° excitation/emission arrangement using a phase-sensitive detector for operation in ambient light. Analysis was performed using custom software written in LabVIEW. Raw fluorescence signal was normalized using exponential background removal method and temperature-shifted to address temperature offset variation between measurements (Palais R et al., Methods in Enzymology, 454. Chapter 13. (2009)) (see FIG. 17).

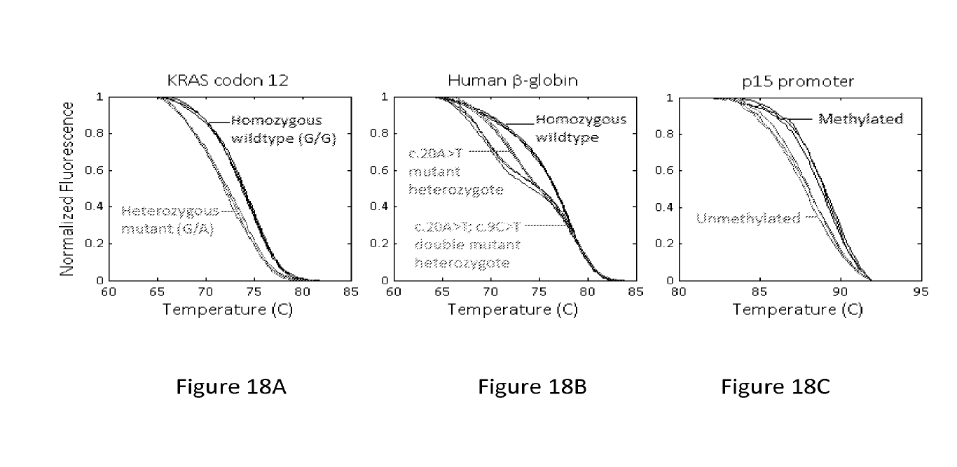

We demonstrated the capability of our system using synthetic DNA molecules representing three sets of biomarkers. Equimolar mixtures of wildtype and variant target strands were hybridized to wildtype probe strand. Melt curves obtained from each set clearly present distinctive melting curve patterns for different genotypes (FIGS. 18A-18C). Melt curve profiles for Kras and beta-globin both show clear separation between homozygous wildtype and mutant heterozygote, marked by higher melting temperature of the wildtype. In the beta-globin set, double mutation heterozygote is clearly marked by a double transition, indicating a large decrease in melting temperature with double mutant heteroduplexes. Melt curve profiles of the synthetic CDKN2B MSP product also show clear difference between methylated and unmethylated sequences. The unmethylated sequence presents lower melting temperature because the unmethylated cytosines are converted to uracils, resulting in lower GC content. This example demonstrates genotyping capability using a simple droplet-based portable apparatus.

The embodiments illustrated and discussed in this specification are intended only to teach those skilled in the art the best way known to the inventors to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A fluidic cartridge for biochemical assays, comprising:
a cartridge body defining a first droplet region and a second droplet region with a droplet restraining barrier therebetween, said droplet restraining barrier having a gap between said first and said second droplet regions;
a first droplet dispensed in said first droplet region, said first droplet comprising a plurality of magnetic particles dispersed therein; and
a second droplet disposed in said second droplet region,
wherein said plurality of magnetic particles are sufficiently small to be drawn through said gap between said first and second droplet regions when compelled by an applied magnetic field, and
wherein said first droplet is restrained by said restraining barrier while said plurality of magnetic particles are drawn through said gap.

2. The fluidic cartridge according to claim 1, wherein said plurality of magnetic particles is a plurality of silica superparamagnetic particles.

3. The fluidic cartridge according to claim 1, wherein said first droplet comprises a binding solution for promoting binding of said plurality of magnetic particles to at least one of molecules of organisms when a sample is added to said first droplet while in operation.

4. The fluidic cartridge according to claim 1, wherein said first droplet comprises at least one of a washing solution or a solution for performing DNA amplification reactions.

5. The fluidic cartridge according to claim 1, further comprising oil contained within said cartridge body to enhance storage and containment of said first and second droplets within said fluidic cartridge.

6. The fluidic cartridge according to claim 5, wherein said first and second droplets are preloaded in said cartridge containing chemicals and reagents for performing biochemical assays, said droplet being covered with said oil and said cartridge body defining topographical features to help maintain said droplets in position during storage.

7. The fluidic cartridge according to claim 1, wherein said cartridge body further defines a third droplet region adjacent to said second droplet region with a second droplet restraining barrier therebetween, said second droplet restraining barrier having a gap between said second and said third droplet regions,
wherein said cartridge body further defines a fourth droplet region adjacent to said third droplet region with a third droplet restraining barrier therebetween, said third droplet restraining barrier having a gap between said third and said fourth droplet regions.

8. The fluidic cartridge according to claim 7, further comprising a third droplet dispensed in said third droplet region and a fourth droplet dispensed in said fourth droplet region,
wherein said first droplet comprises a binding solution for promoting binding of said plurality of magnetic particles to at least one of molecules of organisms when a samples is added to said firth droplet while in operation,
wherein said second and third droplets comprise first and second washing solutions, respectively, and
wherein said fourth droplet comprises a solution for performing polymerase chain reactions.

9. The fluidic cartridge according to claim 8, wherein said cartridge body further defines a well in fluid connection with said fourth droplet region.

10. The fluidic cartridge according to claim 1, further comprising a sample splitting section.

11. The fluidic cartridge according to claim 10, wherein said sample splitting section comprises a plurality of electrodes that are arranged to split a sample droplet into a plurality of split droplets by electro-wetting on dielectric, and wherein said sample splitting section is configured such that at least one of said plurality of split droplets is in fluid connection with said first droplet.

12. The fluidic cartridge according to claim 1, further comprising a heating element at least one of attached to or integrated into said cartridge body.

13. The fluidic cartridge according to claim 1, further comprising a magnetic component at least one of attached to or integrated into said cartridge body.

14. A biochemical assay system, comprising:
a stage adapted to receive a fluidic cartridge; and
a magnetic control assembly comprising a magnet,
wherein said magnet of said magnetic control assembly is movable to direct motion of magnetic particles contained within said fluidic cartridge.

15. The biochemical assay system according to claim 14, further comprising a heating system arranged to controllably heat a droplet position of said fluidic cartridge.

16. The biochemical assay system according to claim 14, further comprising a fluorescence detection system adapted to be arranged to detect fluorescent light from a sample in said fluidic cartridge while in operation.

17. The biochemical assay system according to claim 14, wherein said fluorescence detection system utilizes a phase-sensitive configuration and allows fluorescence detection in an ambient light configuration.

18. The biochemical assay system according to claim 14, further comprising a fluidic cartridge secured for processing by said stage, said cartridge comprising:
a cartridge body defining a first droplet region and a second droplet region with a droplet restraining barrier therebetween, said droplet restraining barrier having a gap between said first and said second droplet regions;
a first droplet disposed in said first droplet region, said first droplet comprising a plurality of magnetic particles dispersed therein; and
a second droplet disposed in said second droplet region,
wherein said plurality of magnetic particles are sufficiently small to be drawn through said gap between said first and second droplet regions when compelled by an applied magnetic field, and
wherein said first droplet is restrained by said restraining barrier while said plurality of magnetic particles are drawn through said gap.
